(19) United States
(12) Patent Application Publication
(10) Pub. No.: US 2020/0107850 A1
(43) Pub. Date: Apr. 9, 2020

(54) ENDOSCOPIC SLEEVE BLADE

(71) Applicant: A.M. SURGICAL, INC., Smithtown, NY (US)

(72) Inventors: Romi MIRZA, Smithtown, NY (US); Ather MIRZA, Smithtown, NY (US)

(21) Appl. No.: 16/155,117

(22) Filed: Oct. 9, 2018

(51) Int. Cl. A61B 17/32; A61B 1/317; A61B 1/313

(52) U.S. Cl. CPC A61B 17/320016; A61B 1/317; A61B 2017/32004; A61B 17/320036; A61B 1/3137

(57) ABSTRACT

An endoscopic surgical blade is disclosed. The blade is molded to an endoscopic sleeve. The endoscopic sleeve blade is for use in endoscopic surgery by insertion of the sleeve into an operating site. The sleeve is hollow and allows the insertion of an endoscope for viewing of the surgical procedure. Methods for using the sleeve blade and a kit containing the sleeve blade are also disclosed.

### FIELD

The present application relates to medical devices and, in particular, to a surgical blade for endoscopic operations.

### BACKGROUND

Endoscopic surgery is a minimally invasive surgical procedure that is performed through small incisions or natural body openings. An endoscopic procedure typically involves use of specialized devices and remote-control manipulation of instruments with indirect observation of the surgical field through an endoscope or similar device. Comparing to open surgery, endoscopic surgery may result in shorter hospital stays, or allow out-subject treatment. There is a need to further improve the design of the instrument while reducing the cost.

The present application provides a solution that reduces both cost and waste, while simplifying endoscopic surgical procedures by reducing the number of steps and components required.

### SUMMARY

One aspect of the application relates to an endoscopic sleeve blade comprising: a tubular body comprising a hollow passageway with a distal end and a proximal end, and a blade assembly comprising a blade secured between an upper guide arm and a lower guide arm, wherein the blade assembly is positioned at the distal end of the hollow passageway.

In one embodiment, the lower guide arm extends beyond the blade further than the upper guide arm and. In another embodiment, the angle of the cutting surface of the blade relative to the lower edge of the blade is between about 35 degrees and about 45 degrees. In another embodiment, the lower guide arm extends below and beyond the blade. In a particular embodiment, the blade is secured by molding into the endoscopic sleeve. In another embodiment, the height of the blade as measured from the top edge to the bottom edge is between about 1.5 mm and about 3.5 mm. In another embodiment, the height of the blade as measured from the top edge of the blade to the bottom edge of the blade is about 2.7 mm. In another embodiment, the space between the blade and the distal end of the hollow passageway is 0.04-0.1 inches. In another embodiment, the space between the blade and the distal end of the hollow passageway is 0.06 inches.

In another embodiment, the upper guide arm extends above and beyond the blade. In a further embodiment, the upper guide arm and the lower guide arm extend beyond the blade, in another embodiment, the upper guide arm is wider than the lower guide arm. In another embodiment, the distance between the upper guide arm and the lower guide arm is 0.04-0.1 inches. In another embodiment, the distance between the upper guide arm and the lower guide arm is 0.08 inches.

A further aspect of the application is a method for performing a uniportal endoscopic surgical procedure on a subject in need thereof, comprising: a) establishing an entry portal at a location in the proximity of an operation site to establish an entry portal; b) inserting an endoscope into an endoscopic sleeve blade for endoscopic surgical procedures, wherein the endoscopic sleeve blade comprises: an endoscopic sleeve with a distal end and a proximal end that comprises a blade secured to the endoscopic sleeve, wherein the endoscopic sleeve comprises a tubular body comprising a hollow passageway with a distal end and a proximal end, and an upper guide arm and a lower guide arm positioned at the distal end of the hollow passageway, wherein the blade is secured between the upper guide arm and the lower guide arm; c) introducing the distal end of the endoscope/endoscopic sleeve blade combination into the entry portal and advancing the combination a predetermined distance relative to the operation site; d) using the endoscope inserted into the endoscopic sleeve blade for direct visualization of anatomic structures surrounding the endoscopic sleeve blade and positioning of the blade at the operative site; e) advancing the endoscopic sleeve blade so that the blade is in contact with a target tissue at the operation site; f) operatively engaging the target tissue with the blade while advancing the latter under direct visualization through the endoscope so as to perform a desired operative procedure on the target tissue; g) withdrawing the endoscope/endoscopic sleeve 10 blade combination from the operation site through the entry portal.

Another aspect of the application is an instrument kit for implementing an endoscopic surgical procedure comprising: an endoscopic sleeve blade comprising: a tubular body comprising a hollow passageway with a distal end and a proximal end, and a blade assembly comprising a blade secured between an upper guide arm and a lower guide arm, wherein the blade assembly is positioned at the distal end of the hollow passageway, and instructions for using the endoscopic sleeve blade.

### BRIEF DESCRIPTION OF THE DRAWINGS

The present application can be better understood by reference to the following drawings. The drawings are merely exemplary to illustrate certain features that may be used singularly or in combination with other features and the present application should not be limited to the embodiments shown.

FIG. 1 illustrates a three-dimensional view of one embodiment of the endoscopic sleeve blade.

FIG. 2 illustrates a side-view of one embodiment of the endoscopic sleeve blade.

FIG. 3 illustrates a front view from the distal end of the endoscopic sleeve blade showing the upper guide arm, lower guide arm, blade, and tubular body with hollow passageway.

FIG. 4 illustrates a top view of one embodiment of the endoscopic sleeve blade.

FIG. 5 illustrates a three-dimensional view of the blade,

FIG. 6 illustrates a side-view of the blade.

FIG. 7 illustrates a cross-sectional view of the blade from line A-A of FIG. 6.

FIG. 8 illustrates a front view of the blade alone.

### DETAILED DESCRIPTION

The following detailed description is presented to enable any person skilled in the art to make and use the object of this application. For purposes of explanation, specific nomenclature is set forth to provide a thorough understanding of the present application. However, it will be apparent to one skilled in the art that these specific details are not required to practice the subject of this application. Descriptions of specific applications are provided only as representative examples. Such description is non-limiting and the scope of the application is intended to encompass all equivalents, alternatives, and modifications, either generally known, or incorporated here. The present application is not intended to be limited to the embodiments shown, but is to be accorded the widest possible scope consistent with the principles and features disclosed herein. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. In the description, relative terms such as “front,” “back,” “up,” “down,” “top,” “bottom,” “upper,” “lower,” “distal,” and “proximal” as well as derivatives thereof, should be construed to refer to the orientation as then described or as shown in the drawing figure under discussion. These, relative terms are for convenience of description and normally are not intended to require a particular orientation. Terms concerning attachments, coupling and the like, such as “connected,” “mounted,” and “attached,” refer to a relationship wherein structures are secured or attached to one another either directly or indirectly through intervening structures, as well as both movable or rigid attachments or relationships, unless expressly described otherwise.

As used in this specification and the appended claims, the singular forms “a,” “an” and“the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it is understood that the particular value forms another embodiment. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

As used herein, the term “subject” refers to an animal. In some embodiments, the animal is a mammal. In further embodiments, the mammal is a human.

As used herein, the term “carpal tunnel syndrome” is usually characterized by numbness in the thumb, index, middle and ring fingers resulting from pressure being exerted on the median nerve inside the carpal tunnel, interfering with the function of such median nerve. This may readily manifest itself as a pain radiating as far as the shoulders and neck of the patient, resulting in impaired grasping ability by the hand and loss of sleep. This physical phenomenon is commonly the result of repetitive work and motions being carried out with the hand over lengthy periods of time, and is experienced by more ever younger people.

In essence, the carpal tunnel is formed by an arch of the eight wrist bones, spanned on its palmar surface by the transverse carpal ligament, the flexor retinaculum. The carpal tunnel functions as a large mechanical pulley to provide the appropriate moment arms for the digital flexor tendons as they pass through the tunnel. The tendons can then transmit force out into the fingers and impart only an appropriate amount of tension to develop torque at the level of the wrist.

Within the carpal tunnel, these tendons are lubricated and nourished by two synovial membranes—the radial and the ulnar bursa. The median nerve also shares the carpal tunnel, then branches out to provide sensory innervation to the palmar surfaces of the thumb, index, long and a portion of the ring finger. In addition, a small motor branch of the median nerve supplies the thenar muscles, which are responsible for lifting the thumb into opposition with the fingers.

### Surgical Procedure

To treat carpal tunnel syndrome by implementing carpal tunnel release has often involved the forming of a lengthy incision, up to 8 cm in length across the palm from the wrist to the middle thereof, resulting in an unsightly scar requiring division of all anatomical structures between the skin and the flexor retinaculum; i.e. the transverse carpal ligament. This created the potential for inadvertently cutting or injuring the palmar cutaneous nerve. Moreover, the patient normally encountered significant postoperative pain and discomfort, weakness of grip and pinch strength because of pillar infraction and the excessively lengthy extent of the incision. Such open surgery not only normally left the patient with a cosmetically unsightly scar extending from the wrist to the center of the palm, as mentioned hereinbefore, but also necessitated a lengthy and painful convalescence for the patient, whereby this convalescent period frequently caused the hand to be incapable of any significant physical work or manipulation for many weeks and even months, thereby effectively rendering the patient incapable of carrying out any meaningful work with the operated on hand and resulting in considerable financial losses being sustained by the patient.

An exemplary endoscopic procedure is directed to the severing of the flexor retinaculum or transverse carpal ligament through an endoscopic surgical procedure in which there is effected, by means of a uniportal or single incision, a palmar subligmentous endoscopic carpal tunnel release technique. This surgical procedure only requires the formation of a single and relatively small entry portal or incision in the palm proximate the distal side of the flexor retinaculum, thereby reducing any postoperative symptoms of the patient with only a cosmetically appealing scar formed on the palm, while eliminating the need for a second portal or incision proximate the wrist of the patient; and concurrently avoiding injury to the pal mar arch and branches of the median nerve. Moreover, the endoscopic instrument employed in implementing the surgical method utilizes a cutting device which is mounted on a scope insertable through a cannula which has been initially inserted to extend beneath the flexor retinaculum from the distal side of the flexor retinaculum or transverse carpal ligament, upon the formation of a passage beneath the flexor retinaculum, after hyperextending of the hand, by the preceding insertion and manipulation of a curved dissector. Thereafter, the dissector is removed and the cannula and an obturator which is contained therein are inserted through the incision into the previously formed passage beneath the flexor retinaculum. The cannula of the surgical instrument has the obturator withdrawn therefrom, and in place of the latter, a scope is inserted into the cannula which enables unhindered and unobstructed visualization of the operating, site and of the flexor retinaculum.

The scope is then withdrawn from the cannula, and the same scope or another scope with a cutting blade mounted at the leading end thereof inserted into and advanced through the cannula towards the flexor retinaculum. Severing of the latter is then effected by the cutting blade while affording an unhindered view of the operating site through the scope, thereby resultingly dramatically reducing or even completely eliminating the risk of any injury being sustained by tissue and nerves in the vicinity of the operating site; for example, such as the median nerve. This particular unhindered visualization of the operating site also enables the surgeon to exercise an improved degree of control over the possibly single-handed manipulation of the endoscopic instrument and cutting blade.

The cannula of the endoscopic instrument, which contains the obturator which is initially employed to be advanced beneath the flexor retinaculum or transverse carpal ligament subsequent to withdrawal of the curved dissector, may be provided with lateral or sideways wing-like or flange-like protrusions of curvilinear configurations which, in conjunction with an upwardly curving tip of the obturator projecting forwardly of the leading end of the cannula, is adapted to displace any tissue, or such as the media nerve, out of the path of the obturator and cannula as is being advanced; in effect, through essentially a sideways or lateral “shoving” action, thereby preventing any potential damage to such displaced tissue and nerve during the subsequent cutting procedure by maintaining such tissue well out of the way. Moreover, the leading tip of the obturator by being curved slightly upwardly towards the lower surface of the flexor retinaculum is also adapted to remove or dislocate any possible tissue or fascia located close to the surface of the flexor retinaculum and to ensure that the cannula and, resultingly, the subsequently inserted cutting blade are located as closely as possible to the flexor retinaculum.

The foregoing endoscopic surgical methods are still further simplified by use of the device described herein.

### The Device

The present application is directed to a simplified device for the performance of endoscopic procedures. In some embodiments, an endoscopic procedure includes division of a target tissue. The device incorporates a cutting blade and guide arms, which function to direct a target tissue to the cutting blade into a single unit that mounts onto the front of an endoscope. The guide arms serve the additional function of protecting tissues above and below the target tissue from cutting.

In some embodiments, the device can be handheld or held by another device.

In some embodiments, an endoscope suitable for use with the present device is adapted to interface with a portable monitoring device. In some embodiments, the portable monitoring device is a laptop computer, tablet or smart phone. In some embodiments, the interface is by direct wiring, or wirelessly, including, but not limited to WiFi or Bluetooth.

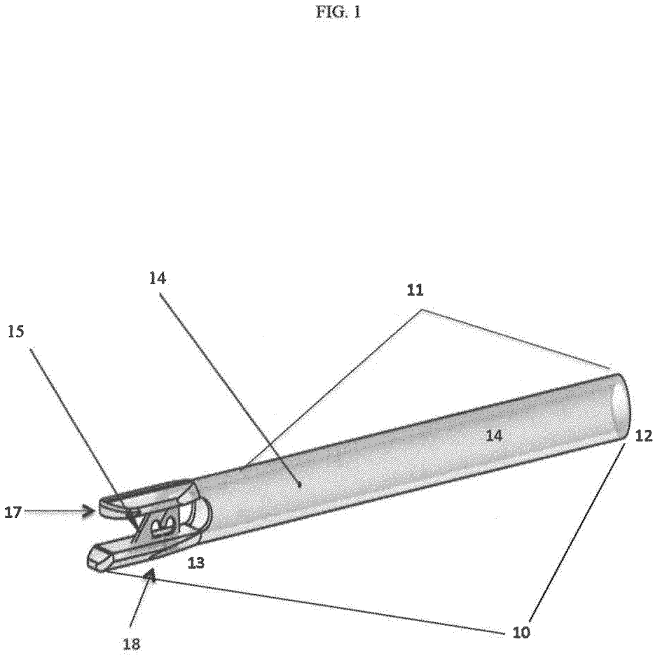

FIG. 1 shows one aspect of the present application relates to an endoscopic sleeve 10 that is molded to a blade 15 for manually cutting connective tissue; the sleeve 10 is specifically designed for endoscopic surgical procedures. The sleeve 10 has a tubular body 11 with a proximal end 12 and a distal end 13, a hollow passageway 14 extending longitudinally from the beginning of the proximal end 12 to distal end 13, the hollow passageway ending before the position of the blade 15, the blade 15 integrally molded near the distal end 13 of the sleeve 10 and positioned between an upper guide arm 17 and a lower guide arm 18. The upper guide arm 17, lower guide arm 18 and blade 15 form together a blade assembly 19. The sleeve 10 then comprises the tubular body 11 and blade assembly 19. The upper guide arm 17 is attached to the top edge of the blade 15 which ends at the trailing end of the blade cutting surface 22. The lower guide arm is attached to the lower edge of the blade 15 which ends at the leading end of the blade cutting surface 22. The upper guide arm 17 and lower guide arm 18 may either be integrated with the tubular body 11 or, alternatively, be separately attachable and detachable from the tubular body 11 as part of the blade assembly 19. The hollow passageway 14 starts at the very beginning of the proximal end of the tubular body 11 so that an endoscopic instrument with a matching structure can be inserted into the sleeve 10 from the proximal end of the tubular body. The hollow passageway 14 ends at a short distance from position of the blade 15 near the distal end of the tubular body 11 so that the endoscope can view the cutting edge of the blade 15. The tubular body 11 is made from a transparent material and has an inner diameter large enough for an endoscope to pass through. In certain embodiments the lower guide arm 18 extends beyond the blade 15 further than the upper guide arm 17 while the upper guide arm 17 is wider than the lower guide arm 18. The sleeve 10 allows a controlled movement of a surgical blade 15 that is held in position by the upper guide arm 17 and the lower guide arm 18.

In order to insure that the blade 15 is consistently joined to the sleeve 10 in the same location, during manufacture the blade 15 is molded into the sleeve 10. In a particular embodiment, the strength of the weld is tested by applying torque to the unit, for example about 10 in-lbs of torque.

In various embodiments, transparent materials, different colored materials or non-transparent materials may be used to form the endoscopic sleeve 10. In one embodiment, the sleeve 10 is made from a transparent plastic material. As used hereinafter, the term “transparent plastic material” refers to a polymer material that has a light transmission rate equal to, or greater than, 80%. Preferably, the transparent plastic material has a light transmission rate equal to, or greater than, 90%.

The transparency of the sleeve 10 wall makes it possible to observe the anatomical structure around the insertion path of the sleeve 10 with an endoscope. In addition, the plastic sleeve 10 is lightweight and can be made by injection molding to reduce cost. The transparent plastics used in the present application should have good impact resistance and abrasion resistance. In one embodiment, the transparent plastics may be coated with a cover layer such as alumina or diamond like carbon, to improve abrasion resistance. The tubular body 11 may further contain observation holes for better identification of the surrounding tissue. In one embodiment, the observation holes are oblong openings on the sides of tubular body 11.

Examples of transparent plastics include, but are not limited to, polyacrylate such as polymethylmethacrylate, polycarbonate, polystyrene, glycol modified polyethylene terephthalate, and cellulose acetate butyrate. Transparent plastics are commercially available under the trade names of Acrystex®, NAS®, Empera®, Kibiton®, Zylar®, Zytel®, etc.). The transparent sleeve 10 can be used in combination with a variety of surgical instruments.

The transparent sleeve 10 of the present application can be inserted into the tissue through a small opening and advanced to a surgical site, thus forming a passageway towards the surgical site. The passageway allows the insertion of an endoscope and other instruments to the surgical site without further damages to the surrounding tissues. The transparent sleeve 10 also allows endoscopic examination of the surrounding anatomical structures without any movement of the sleeve 10. The sleeve 10 provides improved visualization of the target anatomical structure and control over the blade 15. The sleeve 10 is lightweight and can be produced at low cost. A device as embodied by the present application has an advantage for the practitioner and the subject in that it can be provided sterile as a single use and disposable device. The sleeve 10 can be used in endoscopic surgical procedures such as carpal tunnel release, cubital tunnel release, plantar fascia release, lateral release for patella realignment, release of the extensor tendons for lateral epicondylitis (tennis elbow), release of the posterior and other compartments of the leg, and the forearm fascial release for fascial compartment syndrome.

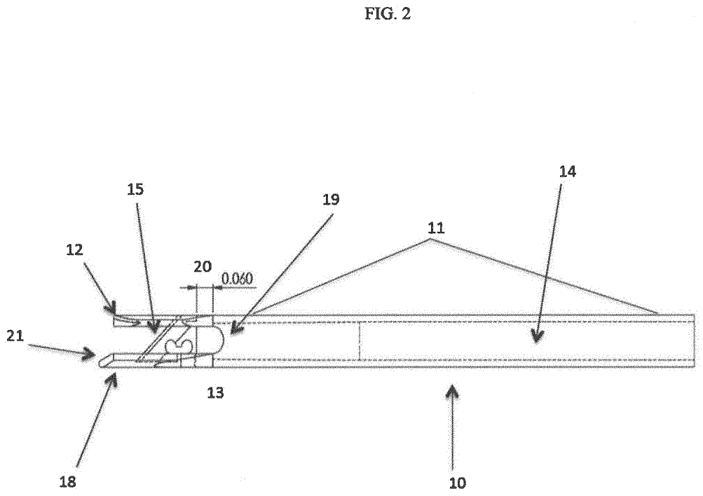

FIG. 2 shows in one embodiment of a surgical blade 15 which is designed to be molded into the sleeve 10, is molded near the leading distal end 13 of sleeve 10. The hollow passageway 14 has an opening at the distal end of the tubular body 11, which is spaced at a short distance behind the knife blade 15, or any cutting or rasp instrument for removing tissue, such as a “curtain” of tissue, which is provided instead of the knife blade 15. An endoscope may be advanced through the hollow passageway 14 until the leading end of the endoscope enters the space 20 behind the blade 15. The space 20 behind the blade 15 and the distal end of the hollow passageway may be 0.04-0.1 inches, preferably 0.06 inches.

The endoscope has its leading end appropriately positioned in proximity relative to the blade 15, and is maintained in position between the upper guide arm 17 and lower guide arm 18; the endoscope can rest on either guide arm as the procedure may require.

In a particular embodiment, the length of the sleeve 10 is from about 100 mm to about 140 mm, preferably from about 114.3 mm to about 119.4 mm. In another embodiment, the length of the sleeve 10 is about 116.8 mm. The sleeve 10 may be 3 inches in length from distal end to proximal end, preferably 2.23 inches; the tubular body of the sleeve 10 may be 1-2.5 inches in length, preferably 1.8 inches. The upper guide arm 17 may be 0.02-0.04 inches ire height, preferably 0.032 inches. The lower guide arm 18 may be 0.03-0.05 inches in height, preferably 0.04 inches. The length of the lower guide arm 18 may be 0.3-0.5 inches, preferably 0.415 inches. The length of the upper guide arm 17 may be 0.3-0.5 inches, preferably 0.37 inches. The sloping lip 21 of the lower guide arm 18 at the distal end of the endoscopic sleeve 10 may be twenty to thirty degrees, preferably twenty-five degrees.

In another particular embodiment, the proximal end of the sleeve 10 is flared. In particular, the about 0.2 to about 0.5 most proximal millimeters of the sleeve 10 are flared. In one embodiment, about the 0.38 most proximal millimeters of the sleeve 10 are flared. In another embodiment, the angle of the flare is about 20 to 40 degrees, more preferably about 30 degrees. In still another embodiment, the outer diameter of the flared portion of the sleeve 10 is about 0.25 mm to about 0.45 mm, more preferably about 0.356 mm.

In an alternate embodiment, the sleeve 10 can optionally be marked on the top or side surface with gradations to show the distance to the blade 15 cutting surface 22. For example, major gradations can be made to show each centimeter in distance from the blade 15 cutting surface 22, with minor gradations between them to show each 1, 2, 2.5 or 5 millimeters. While the gradations can be applied to the sleeve 10 by any means known in the art, it is preferable to lasermark the gradations on the sleeve 10 for accuracy and permanence. Additionally, the sleeve 10 can also be marked in a similar manner with additional information, for example on the bottom or a side surface of the sleeve 10. Exemplary markings may include, but are not limited to, a maker's mark, part number, lot number and an indication that the sleeve 10 is intended for only a single use.

In an alternate embodiment, the sleeve 10 may further comprise slots in the proximal end that are positioned on the sides of the sleeve 10, perpendicular to the blade 15 molded into the sleeve 10. The slots and an alignment ring may provide an attachment point for a locking device, in order to mount an endoscope to the sleeve 10.

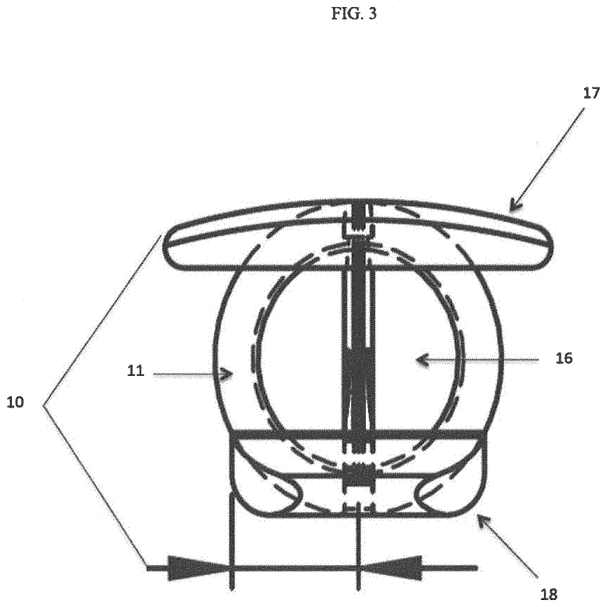

FIG. 3 shows a front view of the tubular body 11 of the sleeve 10 is circular in cross-sectional configuration and has a central aperture 16 to filename passage of an endoscope or other surgical devices through the hollow passageway of the tubular body. The diameter of the cross-sectional configuration may be 0.04-0.1 inches, preferably 0.067 inches. The central aperture 16 is sized to allow passage of such necessary surgical instruments, such as endoscopes, with sufficient clearance. In one embodiment, the central aperture has a diameter of 1-10 mm, preferably 2-8 mm, and more preferably 2-5 mm. The substantially circular central aperture 16 can have a cross-section of any shape, so long as it allows the passage of an endoscope of some type. The outside diameter of the tubular body 11 varies with circumstances but is typically in the range of 2-12 mm, preferably 4-10 mm, and more preferably 4-7 mm. The length of the tubular body 11 also varies with application. For example, the tubular body 11 would have a length of about 5-25 cm; preferably, 12-18 cm; and more preferably, 10-15 cm for carpal tunnel release and cubital tunnel release. The distance between the upper guide arm 17 and the lower guide arm 18 may be 0.04-0.1 inches, preferably 0.08 inches. The lower guide arm 18 may be 0.1-0.2 inches wide at its widest point, preferably 0.13 inches.

In a particular embodiment, the outer diameter of the sleeve 10 is between about 2.8 mm and about 3.6 mm, preferably between about 3.07 mm and about 3.18 mm. In another embodiment, the outer diameter is about 3.12 mm.

The inner diameter of the sleeve 10 is such that an endoscope camera can be inserted into the sleeve 10 in order to show the blade 15 and the target tissue during a procedure. In one embodiment, the inner diameter is between about 2 mm and about 3.5 mm. In another embodiment, the inner diameter 142 is between about 2.76 mm and about 2.87 mm. In a further embodiment, the inner diameter 142 is about 2.81 mm.

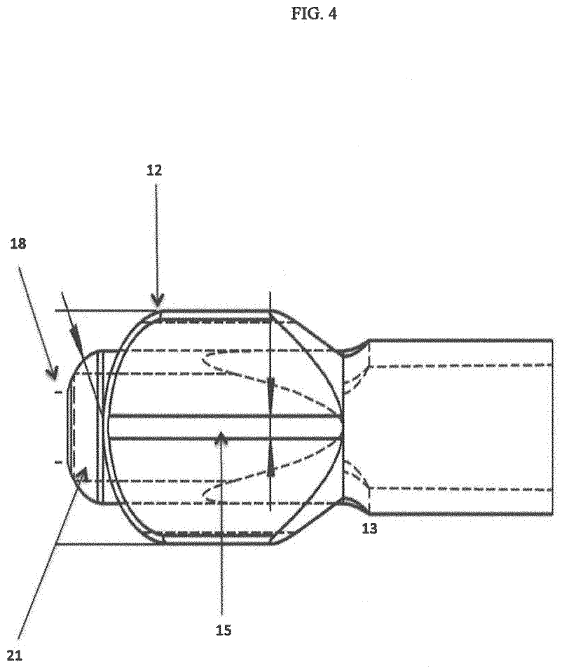

FIG. 4 shows in one embodiment the upper guide arm 17 is wider than the lower guide arm 18. The lower guide arm 18 has a sloping lip that ends at the most distal end of the endoscopic sleeve 10. Both the upper guide arm 17 and lower guide arm 18 are integrated as part of the endoscopic sleeve 10. The lower guide arm 18 may be 0.04-0.1 inches in width at the narrowest distal end of the endoscopic sleeve 10, preferably 0.061 inches in width. The upper guide arm 17 may be 0.1-0.3 inches in width at its widest point, preferably 0.2 inches in width. The blade 15 may be 0.01-0.03 inches in width, preferably 0.02 inches.

In certain alternate embodiments the upper guide arm 17, lower guide arm 18 and molded blade 15 form a detachable or disposable head that may be connected to the tubular body of the endoscopic blade 15 according to any arrangement known to one of ordinary skill in the art (e.g. snap-fit). In this manner, the head may be exchanged for another one which contains a differently shaped or sized blade 15 if so required, e.g., a blade 15 with a crotch in it forming an upper cutting surface and lower cutting surface for the blade 15.

In another alternate embodiment, the upper guide arm 17 and lower guide arm 18, which mount the blade 15 near the distal end 13, may be calibrated along the length thereof so as to provide indication as to the depth to which the instrument is being introduced into the subject towards the surgical site. In this connection, in lieu of the sleeve 10 mounting a blade 15, prior to the use thereof with the endoscope, a sleeve 10 having calibrating markings along the length of the guide arms thereof, which is similar to sleeve 10, may be inserted to the operating site to provide illumination thereof. Upon determination of the appropriate insertion depth to the surgical site by means of the sleeve 10 used for illumination of the site by the endoscope, the sleeve 10 having a molded blade 15 may be substituted therefore. Since the sleeve 10 has the same calibrated markings as the sleeve 10 used for initial illumination of the operating site, this will enable the precise determination of the depth to which the blade 15 molded between upper guide arm 17 and lower guide arm 18 can be inserted into the operating site, thereby preventing any injury due to any excessive penetration past the operating site by the cutting instrument 15.

In certain embodiments, an endoscopic sleeve blade comprises a tubular body comprising a hollow passageway with a distal end and a proximal end, and a blade assembly comprising a blade secured between an upper guide arm 17 and a lower guide arm 18, wherein the lower guide arm 18 extends beyond the blade further than the upper guide arm 17 and wherein the blade assembly is positioned at the distal end of the hollow passageway. In particular embodiments, the upper guide arm 17 extends beyond the blade further than the lower guide arm 18. In other embodiments, the lower guide arm 18 extends beyond the blade further than the upper guide arm 17. In certain embodiments, the upper guide arm 17 extends beyond the blade further than the lower guide arm 18 and the upper guide arm 17 is also wider than the lower guide arm 18. In specific embodiments, the lower guide arm 18 extends beyond the blade further than the upper guide arm 17 and the lower guide arm 18 is also wider than the upper guide arm 17. In particular embodiments, the upper guide and lower guide arm 18 extend beyond the blade for the same distance. In other embodiments, the upper guide and lower guide arm 18 extend beyond the blade for the same distance, and the upper guide arm 17 is also wider than the lower guide arm 18. In further embodiments, the upper guide and lower guide arm 18 extend beyond the blade for the same distance, and the lower guide arm 18 is also wider than the upper guide arm 17.

### The Bade

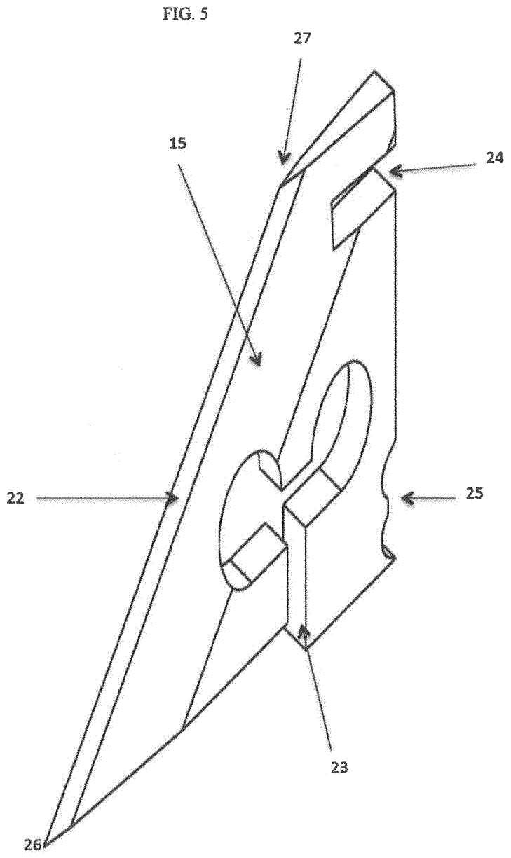

FIG. 5 depicts a three-dimensional view of the blade 15 component of the sleeve 10, showing the cutting surface 22 at the leading end of the blade 15, the transitions 23, 24, 25 where the blade 15 is molded to the upper guide arm 17 and lower guide arm 18 and the taper at the leading end of the blade 15.

In one embodiment, the total length of the blade 15 from the leading point 26 of the cutting surface 22 to the trailing end 27 of the blade 15 is between about 15 mm and about 40 mm. In another embodiment, the total length of the blade 15 from the leading point of the surface to the trailing end of the blade 15 is between about 26.72 mm and about 27.23 mm. In another embodiment the total length of the blade 15 is about 26.97+/−0.26 mm.

In a particular embodiment, the height of the main body of the blade 15 is between about 2 mm and about 3.5 mm. In another embodiment, the height of the main body of the blade 15 is between about 2.54 mm and about 3.04 mm. In another embodiment, the height of the main body of the blade 15 is between about 2.65 mm and about 2.95 mm. In another embodiment, the height of the main body of the blade 15 is about 2.79 mm.

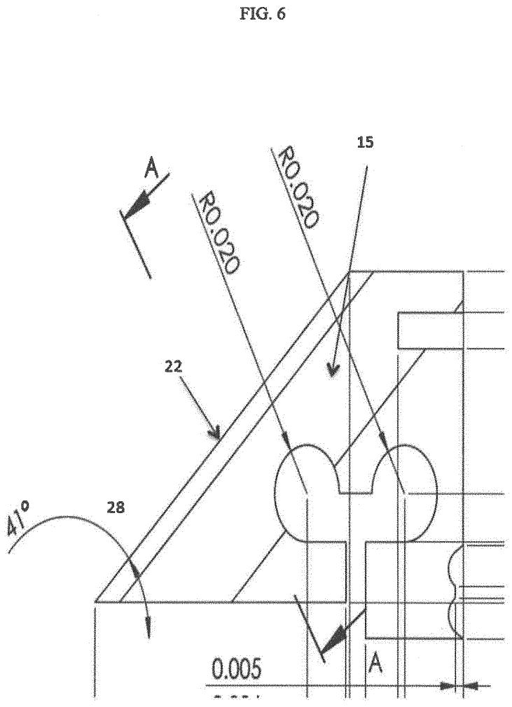

FIG. 6 is a perspective view showing the blade 15 component from the side. In one embodiment, relative to the longer underside of the main body of the blade 15, the angle of the cutting surface 22 is between about 35 degrees and about 45 degrees. In yet another embodiment, the angle is between about 37.5 degrees and about 42.5 degrees. In another embodiment, the angle 28 is about 41 degrees.

In an alternative embodiment, the blade 15 may comprise upper and lower cutting surface 22s on the leading end of the blade 15, which are at an angle to one another and meet at a central crotch. In one embodiment the angle is between about 80 and about 100 degrees. In a preferred embodiment, the angle is between about 85 and about 95 degrees. In a more preferred embodiment, the angle is about 90 degrees. In some embodiments, the grinding of the upper and lower cutting surface 22s where they meet at the crotch has a maximum radius of about 0.65 mm. In a preferred embodiment, the crotch has a maximum radius of about 0.5 mm. In a more preferred embodiment, the crotch has a maximum radius of about 0.38 mm. In some embodiments, the vertical distance between the crotch and the upper edge of the blade 15 is between about 1.5 mm and 2.5 mm. In a preferred embodiment, the vertical distance is about 2.03 mm.

In one alternative embodiment, the upper cutting surface 22 is at an angle to an imaginary line running through the crotch that is parallel to the sleeve 10. In one embodiment the angle is between about 80 and about 100 degrees. In some embodiments, the angle is between about 85 and about 95 degrees. In one embodiment, the angle is about 90 degrees.

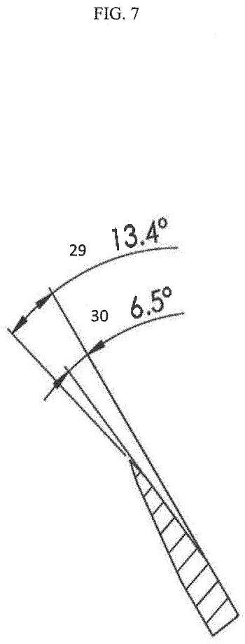

FIG. 7 is a perspective view showing a view of the blade 15 through cross-section A-A in FIG. 6. The angle of the cutting surface 22 at its broadest point 29 relative to the main body of the blade 15 may be 12-15 degrees, preferably 13.4 degrees. The angle of the cutting surface 22 at its narrowest point 30 relative to the main body of the blade 15 may be 5-8 degrees, preferably 6.5 degrees.

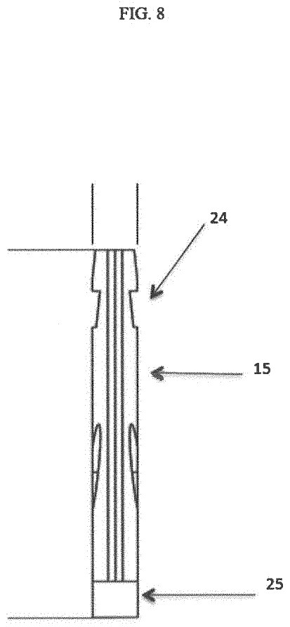

FIG. 8 is a perspective view of the blade 15 from the front. In one embodiment, the vertical height of the blade 15 from the top surface of the blade 15 to the bottom of the cutting surface 22 is between about 2.5 mm to about 5 mm, or about 3.810 mm in one embodiment. In a particular embodiment, the width 136 of the blade 15 is between about 0.4 mm and about 0.7 mm, preferably between about 0.62 mm and about 0.65 mm. In another embodiment, the width 136 of the blade 15 is about 0.64 mm. In one embodiment, the height of the blade 15 at the rear end of the main body of the blade 15 may be 0.14-0.16 inches, preferably 0.152 inches. In one embodiment, the width of the blade 15 at the rear end of the main body of the blade 15 may be 0.013-0.018 inches, preferably 0.015 inches.

The design of the present blade is such that it is usable in endoscopic surgery in a manner that allows the practitioner to extend the blade through the sleeve to the target tissue without damage to surrounding tissue and/or organs. The blade is made from materials commonly used for surgical blades or scalpels, such materials include, but are not limited to, hardened and tempered steel, stainless steel, high carbon steel, titanium, alloys and ceramic.

In particular embodiments, the blade is made from stainless steel. In a further embodiment, the stainless steel is martensitic stainless steel. An exemplary martensitic stainless steel is Bohler-Uddeholm AEB-L martensitic stainless steel. In a still further embodiment, the martensitic stainless steel is heat-treated, in another further embodiment, the stainless steel is 440 A stainless steel. In a particular embodiment, the blade is made from Hitachi GIN-5 SST-MODIFIED 440-A stainless steel. The blade is optionally flash electropolished. The cutting edges are machine finished and must be sharp. In a particular embodiment, the steel of the blade is heat-treated to Rockwell C hardness of about 50-72. In a more particular embodiment, the steel of the blade is heat-treated to Rockwell C hardness of 58-64.

Regarding the blade and blade assembly, the blade assembly provides a stable platform for the blade that fits at the distal end of the tubular body in the sleeve and prevents lateral movement of the blade during deployment. Advancing the sleeve also advances the blade assembly. The advancement of the blade assembly moves the blade into contact with the target tissue. Further advancement of the blade assembly allows the blade to separate the target tissue. In some embodiments, the blade comprises a tab that embeds into the blade assembly. In some embodiments, the blade is secured into the blade assembly using a pin or screw. In such an embodiment, the blade may be replaceable in the blade assembly, allowing the use of blades with different profiles particularly suited for a specific surgical procedure. The blade assembly can be formed of any medically acceptable material, such as a plastic or ceramic, as it may come in contact with body tissues. In a particular embodiment, the assembly is formed of polycarbonate. In some embodiments, the blade assembly may be replaced in the device with any other suitable blade tool that engages with the tubular body of the sleeve.

The use of the present device is exemplified in this application for, but not limited to, division of a pulley or tunnel. Some other non-limiting uses for the present device include, for example, other divisions or partial separation of a tendon or ligament, cutting, dividing, separating or making an incision in connective tissue, muscle, cartilage, membranes, skin, other body tissues or organs or any other use of the device that can be envisioned or carried out by the practitioner. As used herein, the term “practitioner” refers to one of skill in the art or any other user of the present device. In some embodiments, the device can be used for a uniportal endoscopic procedure. In other embodiments, the device can be used for an arthroscopic, laparoscopic, or thoracoscopic procedure. As used herein, “arthroscopic,” “laparoscopic” and “thoracoscopic” procedures fall within the scope of “endoscopic” procedures.

Endoscopic procedures that can be performed with a device of the present application include, but are not limited to, carpal tunnel release, Guyon's canal (or tunnel) release, cubital tunnel release, plantar fascia release, lateral release for patella realignment, release of radial tunnel, release of pronatar teres, release of trigger finger, release of lacertus fibrosus, tendon release, release of the extensor tendons for lateral epicondylitis, release of medial epicondylitis, release of the posterior and other compartments of the leg, forearm fascia release for fascial compartment syndrome, release of fascial compartments in the upper or lower extremities, relieving the compression of a nerve by a ligament pulley or tunnel, and releasing the travel of a ligament or tendon through a pulley or tunnel. Procedures that can be performed with a device of the present application include endoscopic car arthroscopic surgical procedures on the spine, such as discectomy for the treatment of degenerative disc disease, herniated discs, bulging discs, pinched nerves or sciatica. Procedures that can be performed with a device of the present application also include procedures on cranial and facial tissues, as well as fasciotomy release throughout the body. The device of the present application can be used for blood vessel, including vein or artery, harvesting throughout the body, for example to provide blood vessel graft material in conjunction with a coronary bypass procedure or for a reconstructive surgical procedure. Procedures that can be performed with a device of the present application also include endoscopic procedures on the wrist and hand, including the palmar and dorsal sides of the hand. Endoscopic procedures that can be performed with a device of the present application on the hand also include the digits, including the thumb, index finger, middle finger, ring finger and little (pinky) finger. Other examples of endoscopic procedures that can be performed with a device of the present application include, but are not limited to, procedures involving internal tissues or injuries, harvesting of tissues for ex vivo growth; obtaining biopsies; spinal surgery; endonasal surgery; mucosal resection; removal of parasites, cysts or tumors, and foreign body retrieval. Still other examples of endoscopic procedures that can be performed with the device include, but are not limited to, procedures on or within bone, in or around joints or the tendons associated with those joints, as well as any tissue, area or cavity of the body of a subject.

In some embodiments, the present device can be used in the head of a subject. Exemplary procedures in the head include, but are not limited to, nasal surgery, endoscopic sinus surgery, endoscopic pituitary surgery, cranial surgery, endoscopic ear surgery, throat surgery, endodontic surgery and tonsils.

In some embodiments, the present device can be used in the neck of a subject. Exemplary procedures in the neck include, but are not limited to, laryngoscopic surgery, vocal cord surgery, esophageal surgery, thyroid surgery, carotid artery surgery, and brachial plexus surgery.

In some embodiments, the present device can be used in the chest of a subject. Exemplary procedures in the chest include, but are not limited to, endoscopic mediastinal surgery, thoracic surgery, heart surgery, esophageal surgery, and upper gastrointestinal (GI) scoping.

In some embodiments, the present device can be used in a procedure of a finger, hand, foot of a subject.

In some other embodiments, the present device can be used in the abdomen of a subject. Exemplary procedures in the abdomen include, but are not limited to, diagnostic laparoscopy, laparoscopic gastric surgery, laparoscopic liver surgery, laparoscopic pancreatic surgery, laparoscopic nephrectomy and kidney surgery, laparoscopic intestinal surgery, laparoscopic oophorectomy, laparoscopic hysterectomy, laparoscopic urinary bladder surgery, laparoscopic prostate surgery, laparoscopic aortic surgery, laparoscopic appendectomy, laparoscopic colon surgery, endoscopic hysterotomy, endoscopic fetal surgery, endoscopic hernia repair, and endoscopic splenectomy.

In some embodiments, the present device can be used in an upper extremity of a subject. Exemplary procedures in an upper extremity include, but are not limited to, ECTR, ECUTR, endoscopic pronator teres release, forearm fascial compartment release, endoscopic repair of biceps tendon, endoscopic release of lateral and medial epicondylitis, endoscopic release of radial tunnel syndrome, endoscopic surgery of the brachial plexus, endoscopic harvesting of nerve graft, arthroscopy and surgery of wrist, arthroscopy of elbow, arthroscopy and surgery of the carpometacarpal (CMC) joint, arthroscopy and surgery of shoulder, arthroscopy and surgery of acromioclavicular (AC) joint.

In some embodiments, the present device can be used in a lower extremity of a subject. Exemplary procedures in an lower extremity include, but are not limited to, femoral artery surgery, fascia lata release, knee lateral release, endoscopic peroneal nerve release, endoscopic leg fascial compartment release, endoscopic release of gastrocnemius, endoscopic tarsal tunnel release, endoscopic release of Morton's neuroma, endoscopic release of the plantar fascia, arthroscopy of hip, knee and ankle, subtalar joint, and endoscopic harvesting of nerve and tendon graft.

Endoscopic surgical procedures that can be performed with a device of the present application, such as, but not limited to, a ligament or fascia release procedure, can be performed by approaching the target tissue through an incision or body opening on either the proximal or distal side of the target tissue.

In some embodiments, a device of the present application can be used for plastic surgery. A device of the present application is useful for tissue remodeling or the excision of tissue segments, including necrotic tissue.

The device can be used in a clinical setting. The clinical setting can be a hospital, emergency clinic, outpatient clinic, or office, for example. The device can also be used outside the clinical setting, such as, but not limited to, in an emergency situation. The device of the present application can be used by various practitioners including, but not limited to, a physician, surgeon, nurse, nurse practitioner, first responder, paramedic, emergency medical technician, medic, corpsman, technician or caregiver.

### Kit

Another aspect of the present application relates to an instrument kit for implementing an endoscopic surgical procedure. The instrument kit contains a sleeve 10 that has a tubular body 11 with a proximal end 12 and a distal end 13, a hollow passageway extending longitudinally from the beginning of the proximal end 12 to distal end 13, the hollow passageway ending before the position of a blade 15, and upper guide arm 17 and lower guide arm 18 extending from the tubular body, the blade 15 integrally molded into the upper guide arm 17 and lower guide arm 18, the blade 15 positioned near the most distal end of the blade assembly 19, where the tubular body 11 has an inner diameter large enough for an endoscope to pass through.

In one embodiment, the instrument kit further includes an endoscope sized for insertion into the sleeve 10 for direct visualization of an operative site. In another embodiment, the sleeve 10 is made of transparent material. In another embodiment, the instrument kit further includes a second tubular body without a blade molded near the distal end of the second tubular body. In another embodiment, the instrument kit further includes calibrated markings on the upper guide arm 17 and lower guide arm 18 of the sleeve 10. The device can be supplied as part of a kit that includes additional instruments useful with the device such as, but not limited to, scalpel, elevator, dilator, bandages, tape, needles and sutures.

In one embodiment, the instrument kit farther includes an endoscope sized for insertion into the sleeve for direct visualization of an operative site.

In another embodiment, the endoscope is capable to carry a blade at a leading end.

In another embodiment, the instrument kit further includes a blade mountable to the leading end of the endoscope.

In another embodiment, the instrument kit further includes a second endoscope with a blade mounted at a leading end of the second endoscope. The second endoscope is insertable into the sleeve such that the blade protrudes through the opening at the distal end of the tubular body in the sleeve.

In another embodiment, the instrument kit further includes a depth gauge mountable to a leading end of the endoscope.

In another embodiment, the instrument kit further includes a rasp member sized for insertion into the sleeve.

In another embodiment, the instrument kit further includes a locking device capable of locking the endoscope and the sleeve into mutually fixed positions.

In another embodiment, the instrument kit further includes a stop device mountable on the sleeve to prevent excessive penetration at a surgical site by the blade.

In another embodiment, the instrument kit further includes a curved dissector.

The kit may be readily applied to surgical procedures such as for carpal tunnel release; cubital tunnel release, plantar fascia release, lateral release for patella realignment, release of radial tunnel, release of pronatar teres, release of trigger finger, release of lacertous fibrosis, release of the extensor tendons for lateral epicondylitis (tennis elbow), release of medial epicondylitis (golfer's elbow), and release of fascial compartments in the upper and lower extremity. It is also possible to customize the sleeve and endoscopic instruments described above to adapt to other endoscopic surgical procedures.

The following examples serve to illustrate certain embodiments of the application and are not limiting. The contents of all references, patents and published patent applications cited throughout this application, as well as the Figures and Tables are incorporated herein by reference.

### EXAMPLES

### Example 1

### Use of the Endoscopic Sleep Blade (Hereinafter “EndoSleeve Blade) for Endoscopic Carpal Tunnel Release

A single incision is made in the palm proximate to either the proximal side or the distal side of the transverse carpal ligament (TCL) (which side the incision is made is not limiting on the use of the EndoSleeve Blade). A curved dissector is inserted to form a passage beneath the TCL. Once the pathway is created and the dissector removed, the EndoSleeve Blade is introduced into the same pathway. The lower guide arm of the sleeve tip should always stay against the under surface of the TCL and superficial to the flexor tendons and ulnar bursa.

A 4 mm, 30 degree endoscope is then introduced into the sleeve. Visualization of the transverse carpal ligament fibers and fibers of the antebrachial fascia should be visible through the transparent material of the sleeve. If the transverse fibers of the TCL are not clearly seen, the sleeve must be removed and the introduction procedure repeated.

The EndoSleeve Blade should allow for adequate visualization of the median nerve and flexor tendons without the need to rotate the sleeve towards these anatomic structures. Due to variations of anatomy, visual confirmation of these structures may not be possible. If visualization of these structures is inadequate, the surgeon may rotate the sleeve towards the median nerve (radial) and flexor tendons (ulnar) to verify proper sleeve placement.

With a clear view of the transverse fibers of the TCL, as the surgeon observes the monitor, the TCL is divided by advancing the blade molded into the sleeve in a forward direction. As noted above, there is no technical barrier to performing the division of the TCL from either a proximal or distal initial incision in the palm.

Once division is complete and verified, remove the EndoSleeve Blade.

This procedure dramatically reduces the risk of damaging any tissue and nerves, such as the median nerve, in the vicinity of the operating site. It also enables the surgeon to exercise an improved degree of control over the possibly single-handed manipulation of the endoscopic instrument and cutting blade.

### Example 2

### Use of the EndoSleeve Blade for Endoscopic Cubital Tunnel Release

An “X” is placed on both the medial epicondyle and olecranon. A 3-4 cm incision is made along the course of the ulnar nerve at the cubital tunnel between the marked anatomical structures. A dissector is inserted to form a passage beneath the distal and proximal ulnar sheath. Once the distal or proximal pathway is created and the dissector removed, introduce the EndoSleeve Blade into the same pathway. The surgeon should have direct visualization of the ulnar nerve so that the sleeve can be positioned 180 degrees to the ulnar nerve.

A 4 mm, 30 degree endoscope is then introduced into the sleeve. Visualization of the (distal/proximal) ulnar nerve sheath should be visible through the sleeve. If the transverse fibers of the fascia are not clearly seen, the sleeve must be removed and the introduction procedure repeated.

The EndoSleeve Blade should allow for adequate visualization of the ulnar nerve and surrounding tissue without the need to rotate the sleeve towards these anatomic structures. Due to variations of anatomy, visual confirmation of the ulnar nerve may not be possible. If visualization is inadequate, the surgeon may rotate the sleeve towards the ulnar nerve to verify proper sleeve placement.

With a clear view of the transverse fibers of distal/proximal ulnar sheath and no other intervening structures visible within the sleeve, as the surgeon observes the monitor, the sheath is divided by advancing the EndoSleeve Blade.

Once division is complete, and verified, remove the EndoSleeve Blade.

### Example 3

### Uniportal Endoscopic Plantar Fascia Release

In a patient presenting with plantar fasciitis, an incision is made on the medial side of the foot, ahead of the tubercule of the calcaneus.

An endoscopic viewing device is inserted into an endoscopic sleeve blade. The viewing device is advanced into the sleeve and locked in place in relation to the blade assembly.

The endoscopic sleeve blade is introduced into the incision and used to create a plane under the plantar fascia, with the distal end of the blade assembly facing the plantar fascia. The procedure is observed with the viewing device.

The blade is advanced into contact with the plantar fascia. The blade is further pushed forward, dividing the plantar fascia, if the fascia is not completely divided by the first pass of the blade, additional passes may be performed. The cut edges of the plantar fascia and the overlying muscle tissue is visualized through the endoscope. While visualizing the overlying muscle tissue, release may be confirmed by passive manipulation of the ball of the foot. The endoscopic sleeve blade is removed from the incision. The wound is closed and a soft bandage is applied. In some cases, a splint is also applied to immobilize the wrist up to a week.

### Example 4

### Uniportal Arthroscopic Patellar Release

In a patient presenting with lateral patellar tracking disorder, partial division of the lateral retinaculum is indicated to allow the kneecap to shift into the proper position. The lateral retinaculum runs diagonally downward from the patella toward the lateral side of the leg. An incision is made to the lateral side of the lower margin of the patella.

An arthroscopic viewing device is inserted into an endoscopic sleeve. The procedure is observed with the viewing device.

The flexor tendon sheath and the surrounding tissues are again visualized with the arthroscope. The blade is advanced into contact with the lateral retinaculum. The blade is further pushed forward, beginning division of the lateral retinaculum. In some instances, complete division of the lateral retinaculum is not required to allow the patella to shift into the proper position. The endoscopic sleeve blade is retracted. The cut edges of the lateral retinaculum are visualized through the arthroscope.

### Example 5

### Uniportal Endoscopic Trigger Release

Trigger finger is characterized by catching, snapping or locking of the involved finger flexor tendon, associated with dysfunction and pain. Localized inflammation or nodular swelling of said flexor tendon causes a disparity in size between the flexor tendon and the surrounding retinacular pulley system, most commonly at the level of the first annular (A1) pulley. When the subject extends the involved finger, the tendon will “catch” on the pulley, followed by an abrupt popping of the tendon through the pulley. This results in a difficulty flexing or extending the finger and the “triggering” phenomenon.

Typically, a first course of treatment for trigger finger is corticosteroid injections into the tendon sheath to reduce inflammation. When corticosteroid injection is not or no longer effective, surgical division of the A1 pulley is indicated. Conventional surgical techniques for trigger finger release require a fairly large incision over the A1 pulley and spreading of the incision to allow viewing and instrument access. These techniques can require a longer period of recovery than endoscopic methods and have greater levels of post-operative pain due to the incision size and level of manipulation during the procedure. Previous endoscopic techniques for trigger finger release require two incisions, one proximal and one distal to the A1 pulley and the threading of a cannula through the two incisions. An arthroscope is then inserted in the distal end of the cannula, while a cutting tool is inserted in the proximal opening. The cutting tool and arthroscope are then alternately moved forward or backward through the cannula. This does not allow direct visualization of the procedure from the point of view of the cutting tool during the separation of the pulley. Accordingly, the present application fulfills a need in the art for a minimally invasive surgical procedure for the treatment of trigger finger by providing a method for uniportal endoscopic trigger release surgery and an endoscopic surgical blade.

In a patient presenting with trigger finger of the middle or ring finger, a 1 cm incision is made just proximal to the A1 pulley on the distal palmar crease proximate to the affected digit. An elevator is introduced into the incision and used to create a plane superficial to the flexor tendon sheath. The elevator is withdrawn.

An endoscopic sleeve blade inserted therein is introduced into the incision and advanced through the plane created by the elevator. The blade assembly is oriented facing the flexor tendon sheath. An arthroscope is introduced into the endoscopic sleeve and advanced to visualize the A1 pulley and A2 pulley. The arthroscope is withdrawn.

The endoscopic sleeve blade is advanced, dividing the A1 pulley. Having the endoscopic sleeve blade enclosing the arthroscope allows direct visualization of the procedure from the point-of-view of the blade, allowing visualization of the A2 pulley and avoiding damage to the A2 pulley. The arthroscope is used to visualize the cut edges of the A1 pulley, as well as visualization of the underlying flexor tendon. While visualizing the tendon, release of the tendon is confirmed by passive manipulation of the digit through its range of motion. The absence of triggering is confirmed by having the subject flex and extend the affected digit. The arthroscope is withdrawn and the endoscopic sleeve blade is removed from the incision. The wound is closed and a soft bandage is applied. The patient is encouraged to begin early finger motion following surgery and to resume daily activities as tolerated.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

1. An endoscopic sleeve blade comprising:
a tubular body comprising a hollow passageway with a distal end and a proximal end; and
a blade assembly comprising a blade secured between an upper guide arm and a lower guide arm,
wherein the lower guide arm extends beyond the blade further than the upper guide arm, wherein the upper guide arm is wider than the lower guide arm, and wherein the blade assembly is positioned at the distal end of the hollow passageway.

2. The endoscopic sleeve blade of claim 1, wherein the angle of the cutting surface of the blade relative to the lower edge of the blade is between about 35 degrees and about 45 degrees.

3. The endoscopic sleeve blade of claim 1, wherein the lower guide arm extends below and beyond the blade.

4. The endoscopic sleeve blade of claim 1, wherein the blade is secured by molding into the endoscopic sleeve.

5. The endoscopic sleeve blade of claim 1, wherein the height of the blade as measured from the top edge to the bottom edge is between about 1.5 mm and about 3.5 mm.

6. The endoscopic sleeve blade of claim 5, wherein height of the blade as measured from the top edge to the bottom edge is about 2.7 mm.

7. The endoscopic sleeve blade of claim 1, wherein the space between the blade and the distal end of the hollow passageway is 0.04-0.1 inches.

8. The endoscopic sleeve blade of claim 1, wherein the space between the blade and the distal end of the hollow passageway is 0.06 inches.

9. The endoscopic sleeve blade of claim 1, wherein the upper guide arm extends above and beyond the blade.

10. The endoscopic sleeve blade of claim 1, wherein the upper guide arm and the lower guide arm extend beyond the blade.

11. The endoscopic sleeve blade of claim 1, wherein the upper guide arm is wider than the lower guide arm.

12. The endoscopic sleeve blade of claim 1, wherein the distance between the upper guide arm and the lower guide arm is 0.04-0.1 inches.

13. The endoscopic sleeve blade of claim 1, wherein the distance between the upper guide arm and the lower guide arm is 0.08 inches.

14. A method for performing a uniportal endoscopic surgical procedure in a subject in need thereof, comprising:
a) establishing an entry portal at a location in the proximity of a target site;
b) inserting an endoscope into the endoscopic sleeve blade of claim 1 for endoscopic surgical procedures;
c) introducing the distal end of the endoscope/endoscopic sleeve blade combination into the entry portal and advancing the combination a predetermined distance relative to the operation site:
d) using the endoscope inserted into the endoscopic sleeve blade for direct visualization of anatomic structures surrounding the endoscopic sleeve blade and positioning of the blade at the operative site;
e) advancing the endoscopic sleeve blade so that the cutting instrument is in contact with a target tissue at the operation site;
f) operatively engaging the target tissue with the cutting instrument while advancing the latter under direct visualization through the endoscope so as to perform a desired operative procedure on the target tissue;
g) withdrawing the endoscope/endoscopic sleeve blade combination from the operation site through the entry portal.

15. The method of claim 14, wherein establishing an entry portal comprises making an incision.

16. The method of claim 14, wherein the target site is a joint.

17. The method of claim 14, wherein the target site is a ligament, tendon or pulley.

18. The method of claim 14, wherein the target site is fascia.

19. The method of claim 14, wherein the target site is a blood vessel.

20. An instrument kit for implementing an endoscopic surgical procedure comprising:
the endoscopic sleeve blade of claim 1; and
instructions for using the endoscopic sleeve blade.
